(19) United States
(12) Patent Application Publication
(10) Pub. No.: US 2006/0111764 A1
(43) Pub. Date: May 25, 2006

(54) Medical device having a dual fluid circulation structure for thermally affecting tissue

(75) Inventor: John M. Kirkman JR., Kingston, NH (US)

Correspondence Address: John Christopher;Christopher & Weisberg, P.A., Suite 2040, 200 East Las Olas Boulevard, Fort Lauderdale, FL, 33301, US

(73) Assignee: Seacoast Technologies, Inc.

(21) Appl. No.: 10/992,980

(22) Filed: Nov. 19, 2004

(51) Int. Cl. A61F 7/00; A61F 7/12

(52) U.S. Cl. 607/104; 607/105; 607/113

(57) ABSTRACT

A medical device for thermally affecting tissue, having a first heat exchanger, a second heat exchanger at least partially disposed within the first heat exchanger, a first fluid located within the first heat exchanger to at least partially surround the second heat exchanger, and a second fluid circulating through the second heat exchanger. The medical device can be deformable when in contact with tissue, and can further include standoff elements to space or separate the two heat exchangers.

### CROSS-REFERENCE TO RELATED APPLICATION

n/a.

### STATEMENT REGARDING FEDERALLY SPONSORED RESEARCH OR DEVELOPMENT

n/a

### FIELD OF THE INVENTION

The present invention relates to a method and system for thermally affecting tissue.

### BACKGROUND OF THE INVENTION

Researchers and physicians have long recognized the consequences of reduction of body temperature in mammals, including induction of stupor, tissue damage, and death. Application of freezing and near freezing temperatures to selected tissue is commonly employed to preserve tissue and cell (e.g. sperm banks); and application of extreme cold (far below freezing) is effective for tissue ablation. However, localized cooling (not freezing) of tissue has generally been limited to the placement of an “ice-pack” or a “cold compress” on injured or inflamed tissue to reduce swelling and the pain associated therewith. Localized cooling of internal organs, such as the brain, has remained in large part unexplored.

For example, “brain cooling” has been induced by cooling the blood supply to the brain for certain therapies. However, as the effects of the cool blood cannot be easily localized, there is a systemic temperature reduction throughout the body that can lead to cardiac arrhythmia, immune suppression and coagulopathies.

Although attempts have been made to localize cooling of the brain with wholly external devices, such as cooling helmets or neck collars, there are disadvantages associated with external cooling to affect internal tissue. For example, external methods do not provide adequate resolution for selective tissue cooling, and some of the same disadvantages that are associated with systemic cooling can occur when using external cooling devices. Further, internal cooling devices have also been developed, but are often limited in their ability to conform to the shapes of brain tissue targeted for cooling.

In view of the above limitations, it would be desirable to provide a medical device that directly thermally affects tissue and is conformable to surface areas of varying shape.

### SUMMARY OF THE INVENTION

The present invention advantageously provides a medical device that directly thermally affects tissue and is conformable to surface areas of varying shape.

In an exemplary embodiment, the medical device includes a first and second heat exchanger, with the second heat exchanger being at least partially disposed within the first heat exchanger. The medical device further provides for a first fluid to be contained within the first heat exchanger, as well as a second fluid which circulates through the second heat exchanger. Both the first and second fluids can be thermally transmissive fluids which are chilled to below body temperature.

The medical device is constructed from pliant materials, enabling the medical device to deform when in contact with tissue. Further, the pressurization of the fluids implemented in the medical device can be manipulated resulting in varying degrees of pliability of the medical device. The medical device can also include standoff elements on either the first or second heat exchangers, which provide for spacing and separation of the two respective heat exchangers.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates an exemplary cooling system used to perform a medical procedure in accordance with the present invention;

FIG. 2 depicts a cooling structure of the system of FIG. 1;

FIG. 3 illustrates additional details of a cross section of an exemplary cooling structure;

FIG. 4 illustrates additional details of a cross section of an exemplary cooling structure; and

FIG. 5 illustrates an exemplary cooling system used to perform a medical procedure in accordance with the present invention.

### DETAILED DESCRIPTION OF THE INVENTION

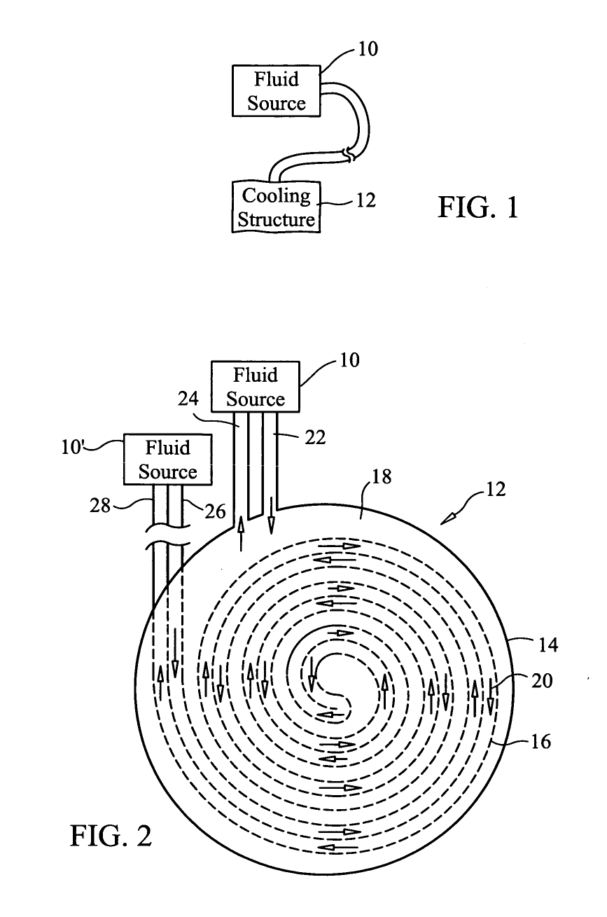

Referring to FIG. 1, the present invention provides for a medical device for thermally affecting tissue, generally including one or more fluid sources 10 connected to a cooling structure 12. Although not shown, the medical device can be included in a system that includes a pump, sensors, a refrigeration unit, and a control system with a user interface to cause fluid to be moved to the cooling structure 12 from the fluid source 10 at a selected rate, temperature, and pressure.

FIG. 2 illustrates an exemplary cooling structure 10 that includes a first heat exchanger 14 and a second heat exchanger 16 at least partially disposed within the first heat exchanger 14. As shown, the first heat exchanger 14 contains a first fluid 18 that partially or completely envelops, flows across, or flows around the second heat exchanger 16. The second heat exchanger 16 contains or provides a passage for a second fluid 20 that circulates within or through the second heat exchanger. Thermal energy is transferable between the first and second fluids 18 and 20, as well as between the first fluid and a point exterior to the cooling structure 12 (e.g., body tissue).

In the illustrated embodiment, the first heat exchanger 14 is provided with an input lumen 22 as well as an output lumen 24, for introducing and evacuating the first fluid 18, respectively, from the first heat exchanger 14. Evacuation of the fluid 18 from the first heat exchanger 14 provides the first heat exchanger and thus the cooling structure 12 with a reduced size as compared to its fluid filled state. However, the first heat exchanger 14 can also be filled to a predetermined volume with fluid 18 and sealed so that a predetermined volume of fluid is permanently trapped within the first heat exchanger 14. When fully deployed, the illustrated cooling structure provides a flexible pad that has diameter significantly greater than its thickness. In an exemplary embodiment, the cooling structure is approximately 60 mm in diameter and 2.5 mm in thickness. The cooling structure can be provided with a greater or lesser diameter depending upon the tissue area to be treated.

Continuing to refer to FIG. 2, the second heat exchanger 16 disposed at least partially or entirely within the first heat exchanger 14 can include an input lumen 26 and an output lumen 28 for transfer of cooling fluid to and/or from a fluid source or a third heat exchanger (not shown) that is separate from the cooling structure 12. The input lumen 26 and output lumen 28 of the second heat exchanger 16 can form a circulation path for the second fluid 20 within the boundaries of the first heat exchanger 14. As shown, the circulation path can be configured such that at least a portion of the circulation path is looped around itself into a coil configuration, but it is not limited to such a configuration.

Additionally, although the input and output lumens of the heat exchangers are shown exiting the periphery of the first heat exchanger 15, they can be configured to enter and exit at other locations, such as the central region on the first heat exchanger. Furthermore, while the input and output lumens of the two heat exchangers are depicted as being separated and leading to separate fluid sources 10 and 10′, they can all be placed in close proximity or a single “bundle” and they can be in fluid communication with a single fluid source 10 or the third heat exchanger (not shown).

The first and second heat exchangers can be made from a pliant material, including various plastic or silicone elastomer materials, or any other material that would allow either or both of the heat exchangers to deform when the cooling structure 12 is placed in contact with tissue. The ready deformability of the first heat exchanger 14 is particularly important as it allows the cooling structure to conform to an uneven or irregular tissue surface, thereby enhancing the ability to thermally affect the tissue. Additionally, either of the heat exchangers can be constructed from thermally transmissive materials having properties that affect thermal conductivity, and the resulting effectiveness to thermally affect tissue by maximizing tissue contact with the heat exchanger. Although the first heat exchanger 14 is shown having an essentially circular shape, it can also be configured as essentially rectangular in shape, or it can be constructed to mirror the shape of a tissue region that will be thermally affected by the medical device of the present invention.

Continuing to refer to FIG. 2, the first fluid 18 that is within the first heat exchanger 14 so as to at least partially surround the second heat exchanger 16 is a thermally transmissive fluid, such as a saline mixture, and it can also be pressurized up to 1.0 psig. However, to maximize pliability of the first heat exchanger 14, the first fluid 18 is preferably kept at a pressure less than 0.677 psig (35 mmHG).

Similarly, the second fluid 20 can be a thermally transmissive fluid, such as a saline mixture, and it can also be pressurized to approximately 20 psig. Because the second heat exchanger 16 is isolated from the tissue to be contacted by the first heat exchanger 14, it can be or become more rigid or less pliant than the first heat exchanger. In operation, the second fluid 20 can be chilled to a temperature below that of the tissue to be affected. In an exemplary application, the second fluid 20 can be cooled to a temperature of −4° C. to −37° C. This in turn leads to a thermal exchange with the first fluid which results in the first fluid 18 being chilled to a temperature below that of the tissue to be affected. The first fluid 18 thus acts as both a distributor of thermal transfer as well as a buffer to prevent localized extreme temperature variation. In other words, the first fluid 18 helps to ensure that the cooling structure 12 presents a tissue contact surface that is substantially uniform in temperature.

Because the first fluid 18 is intended to be cooled by thermal transfer with the second heat exchanger 16, the first fluid does not need to be circulated outside the cooling structure 12, although it can be caused or allowed to circulate within the cooling structure. Therefore, the first fluid 18 does not need to be pressurized, thereby providing it with a pliant characteristic allowing it to conform to a tissue surface, while the second heat exchanger 16 can convey the second fluid 20 at a pressure and rate of circulation sufficient to achieve a desired thermal result at the cooling structure/tissue interface.

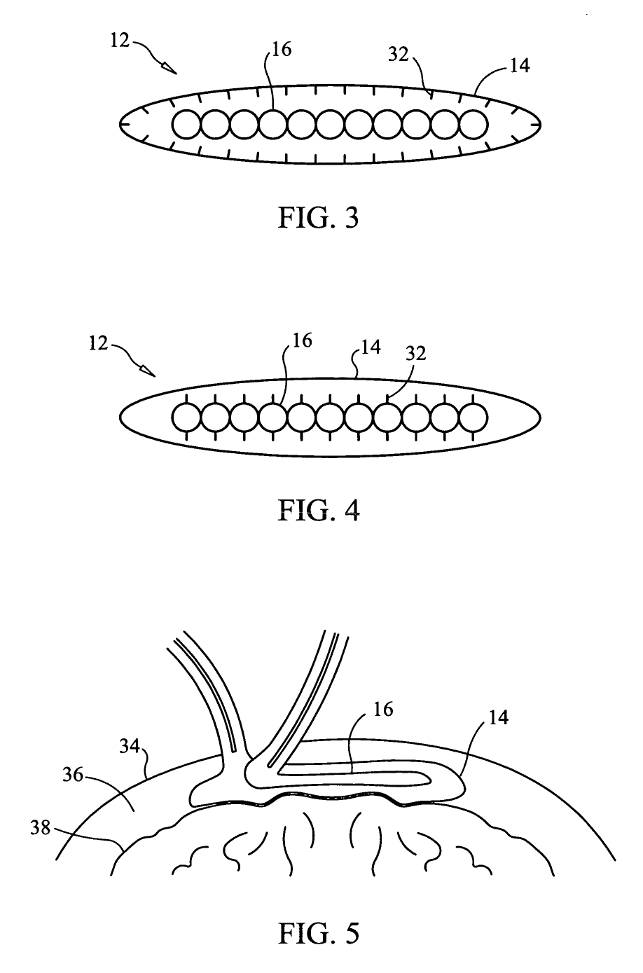

Now referring to FIGS. 3 and 4, cooling structures 12 similar to that of FIG. 2 are shown. In these views spacing elements 32 are illustrated that separate the first heat exchanger 14 from the second heat exchanger 16. The spacing elements 32 can be located on either the first heat exchanger 14 as shown in FIG. 3, or, alternatively, on the second heat exchanger 16, as shown in FIG. 4. The spacing elements 32 provide separation between the inner surface of the second heat exchanger and the outer surface of the first heat exchangers, thereby providing improved isolation of the second heat exchanger 16 within the first heat exchanger 14 to permit and promote flow of the first fluid 18 around the second heat exchanger. Further, the second heat exchanger 16 can be constructed from a material having a density that is less than a density of the first fluid 18, subsequently preventing the portion of the second heat exchanger 16 that is disposed within the first heat exchanger 14 from sinking to the bottom of the first heat exchanger 14 when in use.

Turning now to FIG. 5, a cooling structure is shown in use, wherein a portion of the skull 34 has been removed and the cooling structure placed in the space 36 between the surface of the brain 38 or its covering tissue, the dura, and the interior surface of the skull. Although not shown in the drawings, it is understood that in order to thermally affect tissue other than the brain, the medical device can be placed at other locations in or on a patient. Upon positioning the cooling structure in thermal communication with the tissue to be affected, the first fluid 18 is introduced into the first heat exchanger 14 (if it has not already been sealed therein). By introducing the first fluid 18 at a relatively low pressure, the first heat exchanger 14 can maintain its pliancy and thus conform to any uneven surface of the brain tissue. The second fluid 20 is then circulated within the second heat exchanger 16 at a rate generally predetermined in order to obtain a desired thermal result. Either one of the fluids in the medical device can be chilled below the temperature of the tissue prior to introduction into the heat exchangers. When a particular thermal result is achieved, circulation of the second fluid 20 can cease, and the first fluid 18 can be evacuated from the first heat exchanger 14, thereby facilitating removal of the device from the patient.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical device for thermally affecting tissue, comprising:
a first heat exchanger,
a second heat exchanger at least partially disposed within the first heat exchanger,
a first fluid located within the first heat exchanger to at least partially surround the second heat exchanger, and
a second fluid circulating through the second heat exchanger.

2. The medical device according to claim 1, wherein the first heat exchanger includes an input and an output lumen.

3. The medical device according to claim 1, wherein the first heat exchanger is made from a pliant material.

4. The medical device according to claim 1, wherein the first heat exchanger is made from a thermally transmissive material.

5. The medical device according to claim 1, wherein the first heat exchanger is deformable upon contact with tissue.

6. The medical device according to claim 1, wherein the first heat exchanger has an essentially circular shape.

7. The medical device according to claim 1, wherein the first heat exchanger has an essentially rectangular shape.

8. The medical device according to claim 1, wherein the first heat exchanger includes a plurality of spacing elements to separate the second heat exchanger from the first heat exchanger.

9. The medical device according to claim 1, wherein the second heat exchanger includes a plurality of spacing elements to separate the second heat exchanger from the first heat exchanger.

10. The medical device according to claim 1, wherein the first fluid is a thermally transmissive fluid.

11. The medical device according to claim 1, wherein the first fluid is pressurized to less than 1.0 psig.

12. The medical device according to claim 1, wherein the first fluid is chilled to below body temperature.

13. The medical device according to claim 1, wherein the second heat exchanger is made from a thermally transmissive material.

14. The medical device according to claim 1, wherein the second heat exchanger includes an input lumen and an output lumen.

15. The medical device according to claim 14, wherein the input lumen and output lumen define a fluid circulation path.

16. The medical device according to claim 1, wherein at least a portion of the fluid circulation path is looped around itself in a coil configuration.

17. The medical device according to claim 1, wherein the second fluid is a thermally transmissive fluid.

18. The medical device according to claim 1, wherein the second fluid is chilled to below body temperature.

19. The medical device according to claim 1, wherein the first fluid has a pressure that is substantially less than the pressure of the second fluid.

20. The medical device according to claim 1, wherein the density of the second heat exchanger is less than the density of the first fluid.

21. A medical device for thermally affecting tissue, comprising:
a first heat exchanger including an first input lumen and a first output lumen, wherein the first heat exchanger is deformable upon contact with tissue,
a second heat exchanger at least partially disposed within the first heat exchanger, the second heat exchanger including a second input lumen and a second output lumen, wherein the second input lumen and second output lumen define a fluid circulation path, wherein at least a portion of the fluid circulation path is looped around itself in a coil configuration,
a first fluid located within the first heat exchanger to at least partially surround the second heat exchanger, and
a second fluid circulating through the fluid circulation path, wherein the second fluid has a substantially greater pressure than the first fluid.

22. A method of thermally affecting tissue, comprising the steps of:
positioning a medical device in thermal communication with a tissue, the medical device being comprised of a first heat exchanger, and a second heat exchanger at least partially disposed within the first heat exchanger,
introducing a first fluid into the first heat exchanger,
circulating a second fluid through the second heat exchanger, and
allowing the medical device to thermally affect the tissue.

23. The method according to claim 22, further comprising the step of evacuating the first fluid from the first heat exchanger.

24. The method according to claim 23, further comprising the step of removing the medical device from thermal communication with the tissue.

25. The method according to claim 22, wherein the tissue is brain tissue.
